(19) United States
(12) Patent Application Publication
(10) Pub. No.: US 2016/0199072 A1
(43) Pub. Date: Jul. 14, 2016

(54) BONE REMOVAL UNDER DIRECT VISUALIZATION

(71) Applicant: SMITH & NEPHEW, INC., Memphis, TN (US)

(72) Inventors: Paul Alexander Torrie, Marblehead, MA (US); Laura Mills, Framingham, MA (US); Spencer W. Shore, Hamden, CT (US)

(21) Appl. No.: 14/912,515

(22) PCT Filed: Aug. 19, 2014
(86) PCT No.: PCT/US14/51643

§ 371 Date: Feb. 17, 2016

Related U.S. Application Data
(60) Provisional application No. 61/867,486, filed on Aug. 19, 2013.

(51) Int. Cl. A61B 17/17; A61B 17/16

(52) U.S. Cl. CPC A61B 17/1703; A61B 17/1659; A61B 17/175; A61B 17/1631; A61B 17/1633; A61B 17/1668; A61B 2217/007

(57) ABSTRACT

An approach to removing necrotic bone under direct visualization is provided. To remove bone and to watch it being removed at the same time, one example uses an endoscope and bone removal tool assembled together by a sheath. The sheath includes separate passageways for the endoscope and bone removal tool. A surgeon uses the sheath to insert the endoscope and bone removal tool together into the bone tunnel. The passageways are spaced apart such that an axis of rotation of the bone removal tool is offset from the centerline of the bone tunnel. The endoscope remains in the bone tunnel while the surgeon removes bone with the bone removal tool. Advantageously, the surgeon watches the bone removal process as it is happening making the process less tedious and less time consuming.

### BACKGROUND

Avascular necrosis (AVN) of the femoral neck is a degenerative condition thought to be caused by increased interstitial pressure within the femoral head leading to reduced blood supply to the region and eventually bone necrosis. In the later stages III and IV of the disease, the spherical femoral heads collapses into a non-spherical shape usually with cartilage damage, ultimately requiring a total hip replacement. The challenge is to remove the necrotic bone and replace it with a viable graft or bone graft substitute before the femoral head collapses or cartilage is damaged.

To remove the necrotic bone, while removing as little of the surrounding healthy bone as possible, bone removal is done though the bone tunnel using curettes or buns. Their use is guided, primarily, by tactile feedback and fluoroscopy. One prior approach to bone removal involves a surgeon placing an endoscope down the bone tunnel to see the necrotic bone. In this prior approach, however, the surgeon does not remove bone and view at the same time. Various grafts are then used including autologous bone, allografts, bone graft substitutes, and free vascularized fibula autografts to fill in the void left from removing the necrotic bone. Such grafts are then press fit in or are held in place via mixing with blood, or by screws and plates.

Core decompression is the most common treatment for AVN. The procedure consists of placing a guide-wire from the lateral aspect of the greater trochanter into the femoral head followed by over drilling to form a 9-12 mm diameter bone tunnel, also referred to as a“core decompression tunnel.” The guide-wire is placed by taking multiple orthogonal fluoroscopy images. Challenges with the current core decompression technique include the possibility of drilling through the femoral head and the possibility of leaving some necrotic bone behind preventing a successful graft. What is needed is an approach that places an endoscope into the core decompression tunnel and uses bone removal tools to extract necrotic bone under direct visualization.

### SUMMARY

Described herein are examples of an approach for removing necrotic bone under direct visualization is provided that address the foregoing shortcomings and others as well. In one aspect, at least one example described herein provides a sheath. The sheath includes a body including a proximal end and a distal end, and an inlet disposed at the proximal end of the body through which inflow fluid is provided. The body further includes a first passageway extending, longitudinally, between the proximal and distal ends of the body. The body still further includes a second passageway extending, longitudinally, from the distal end of the body towards the proximal end of the body. The second passageway defines an axis of rotation of a bone removal tool. The first and second passageways are spaced apart such that the axis of rotation of the bone removal tool is offset from the centerline of a bone tunnel. A working length of the body has a diameter smaller than the diameter of the bone tunnel.

In other examples, the sheath may further include one or more of the following, alone or in any combination. In some examples of the sheath, the first passageway is curved with the first passageway and the second passageway spaced apart a first distance at the distal end of the body and spaced apart a second distance greater than the first distance at the proximal end of the body. In other examples of the sheath, at the distal end of the body, the first passageway terminates with a rounded end. In some examples of the sheath, the second passageway is a U-shape trough. In other examples of the sheath, the first passageway and the second passageway are stacked on one another along a lateral axis defined by the inlet.

Some examples of the sheath further include a stop integrally formed with the body. The integrally formed stop includes a first stop surface and an opposed second stop surface. The first and second stop surfaces cooperate with a corresponding bead formed around a shaft of the bone removal tool to limit movement of the bone removal tool along a length of the second passageway. Alternatively, the first and second stop surfaces cooperate with a corresponding bend formed in a shaft of the bone removal tool to limit movement of the bone removal tool along a length of the second passageway.

Other examples of the sheath further include an inclined wall formed between the first passageway and the second passageway. The inclined wall defines a conduit with the wall of the bone tunnel for conducting outflow fluid carrying portions of removed bone.

In another aspect, at least one example described herein provides a system including a sheath and a visualization device received in a first passageway of the sheath. The sheath including a body comprising a proximal end and a distal end and an inlet disposed at the proximal end of the body through which inflow fluid is provided. The body further comprising a first passageway extending, longitudinally, between the proximal and distal ends of the body, and a second passageway extending, longitudinally, from the distal end of the body towards the proximal end of the body. The second passageway defines an axis of rotation of a bone removal tool. The first and second passageways are spaced apart such that the axis of rotation of the bone removal tool is offset from the centerline of the bone tunnel. A working length of the body has a diameter smaller than the diameter of the bone tunnel.

In other examples, the system may further include one or more of the following, alone or in any combination. In some examples, the first passageway of the sheath is curved with the first passageway and the second passageway spaced apart a first distance at the distal end of the body and spaced apart a second distance greater than the first distance at the proximal end of the body. In other examples, the first passageway of the sheath and the visualization device have different cross-sections. The difference in cross-sections defines a conduit for the inflow fluid. In some examples, the visualization device is an endoscope.

Some examples of the system further include a bone removal tool. The bone removal tool includes a shaft and a working end at an end of the shaft. At least a portion of the shaft of the bone removal tool is received in the second passageway of the sheath. The shaft of the bone removal tool may be flexible. The working end of the bone removal tool may include a three-dimensional rasp comprising two cutting edges meeting at a leading point. The leading point meets the wall of the bone tunnel at a 32° angle and contacts bone before the two cutting edges as the working end is rotated. Other examples of the bone removal tool include rotary rasp, articulating rotary curette, articulating planer curette, and rotary wireform.

Other examples of the system further include an inlet port including a first end adapted to mate with the inlet of the sheath and a second end adapted to mate with an inflow fluid source. The inlet port may be in the shape of a handle. Some examples of the second end include a coupling member with a breakaway feature, such that when the second end of the inlet port is being disconnected from an inflow fluid source the coupling member breaks away from the second end.

In yet another aspect, at least one example described herein provides a bone removal tool. The bone removal tool includes a shaft having a length, a portion of which is supported by a passageway of a sheath, and a working end at an end of the shaft. In some examples, the working end has an axis of rotation defined by a second passageway of the sheath and is offset from the centerline of a bone tunnel. The shaft may be flexible. Some examples of the bone removal tool include a bead formed around the shaft. The bead cooperates with a first stop surface and an opposed second stop surface of a stop integrally formed with the sheath. Other examples of the bone removal tool include a bend formed in the shaft. The bend cooperates with a first stop surface and an opposed second stop surface of a stop integrally formed with the sheath.

The working end of the bone removal tool may include a three-dimensional rasp comprising two cutting edges meeting at a leading point. The leading point meets the wall of the bone tunnel at a 32° angle and contacts bone before the two cutting edges as the working end is rotated. Other examples of the bone removal tool include rotary rasp, articulating rotary curette, articulating planer curette, and rotary wireform.

In still yet another aspect, at least one example described herein provides a procedure for removing bone under direct visualization. The procedure includes a) forming a bone tunnel, b) inserting an assembly into the bone tunnel, the assembly including an visualization device received in a first passageway of a sheath and a bone removal tool received in a second passageway of the sheath, c) rotating the bone removal tool about an axis of rotation defined by the second passageway and that is offset from the centerline of the bone tunnel to remove a portion of the bone, and d) viewing the portion of bone being removed while the bone removal tool is being rotated.

In other examples, the procedure may further include one or more of the following, alone or in any combination. Some examples include changing a field of view of the visualization device by rotating the visualization device within the first passageway of the sheath. Other examples include providing an inflow fluid to where bone is being removed through a conduit defined by a difference in cross-section of the visualization device and cross-section of the first passageway of the sheath. Some examples include conducting an outflow fluid carrying portions of removed bone through a conduit defined by the wall of the bone tunnel and an inclined wall formed between the first passageway and the second passageway of the sheath.

Other examples include moving the bone removal tool along a length of the second passageway of the sheath. Some other examples may further include moving a bead formed around a shaft of the bone removal tool between a first stop surface and a second stop surface of a stop integrally formed in the sheath. The bead and the first and second stop surfaces corporate to limit movement of the bone removal tool along the length of the second passageway. Alternative examples may include moving a bend formed in a shaft of the bone removal tool between a first stop surface and a second stop surface of a stop integrally formed in the sheath. The bend and the first and second stop surfaces corporate to limit movement of the bone removal tool along the length of the second passageway.

### BRIEF DESCRIPTION OF THE DRAWINGS

The accompanying drawings, which are incorporated in and form a part of the specification, illustrate examples of the present disclosure and together with the written description serve to explain the principles, characteristics, and features of the disclosure. In the drawings:

FIGS. 1-8 are views of accessing necrotic bone in accordance with examples of an approach for removing necrotic bone under direct visualization.

FIG. 9 is a view of a system for removing necrotic bone in accordance with the approach.

FIGS. 10a and 10b are views of an example of the system removing bone under direct visualization.

FIGS. 11a-11c are views of examples of the system for removing bone under direct visualization.

FIGS. 12a-12c are views of examples of the system with manual and powered bone removal tools.

FIG. 13a-13d are views of the working end of a three-dimensional rasp bone removal tool.

FIG. 14 is a view of an sheath used to remove bone under direct visualization in accordance with the approach.

FIG. 15 is a close-up view of the distal end of the sheath with bone removal tool and endoscope.

FIG. 16 is a sectional view of the distal end of the sheath with bone removal tool and endoscope.

FIGS. 17a-17d are views of a disposable inlet used with an example of the sheath.

FIGS. 18a-18b are views of example tools with curettes that articulate to remove bone under direct visualization.

FIGS. 19a-19c are views of an example tool with a wire form for removing bone under direct visualization.

FIG. 20 is a view of an example integral sheath with a bone removal tool that pivots to remove bone under direct visualization.

FIG. 21 is a view of an example integral sheath with a bone removal tool that expands to remove bone under direct visualization.

### DESCRIPTION

In the following detailed description of the illustrated examples, reference is made to accompanying drawings, which form a part thereof, and within which are shown by way of illustration, specific examples, by which the subject matter can be practiced. It is to be understood that other examples can be utilized and structural changes can be made without departing from the scope of the disclosure.

The particulars shown herein are by way of example and for purposes of illustrative discussion of the examples only and are presented in the case of providing what is believed to be the most useful and readily understood description of the principles and conceptual aspects of the disclosure. In this regard, no attempt is made to show structural details of the subject matter in more detail than is necessary for the fundamental understanding of the disclosure, the description taken with the drawings making apparent to those skilled in that how the several forms of the present disclosure can be embodied in practice. Further, like reference numbers and designations in the various drawings indicate like elements.

An approach for removing necrotic bone under direct visualization is provided. Examples of the approach include accessing necrotic bone and removing necrotic bone. It is noted that some examples of the approach include both accessing and removing necrotic bone while other examples include one or the other. The tools and procedures for accessing necrotic bone are described first. The necrotic bone is accessed through a bone tunnel. As overview, to form the bone tunnel, a surgeon places a guide-wire through bone and into necrotic bone. The surgeon then guides a cannulated drill bit along the guide-wire to drill the bone tunnel through the bone and into the necrotic bone. The surgeon selects a location for the guide-wire based on the shape and size of the necrotic bone. In some examples of the approach, the surgeon uses a three-dimensional guide to assist in placing the guide-wire in the selected (desired) location in the necrotic bone.

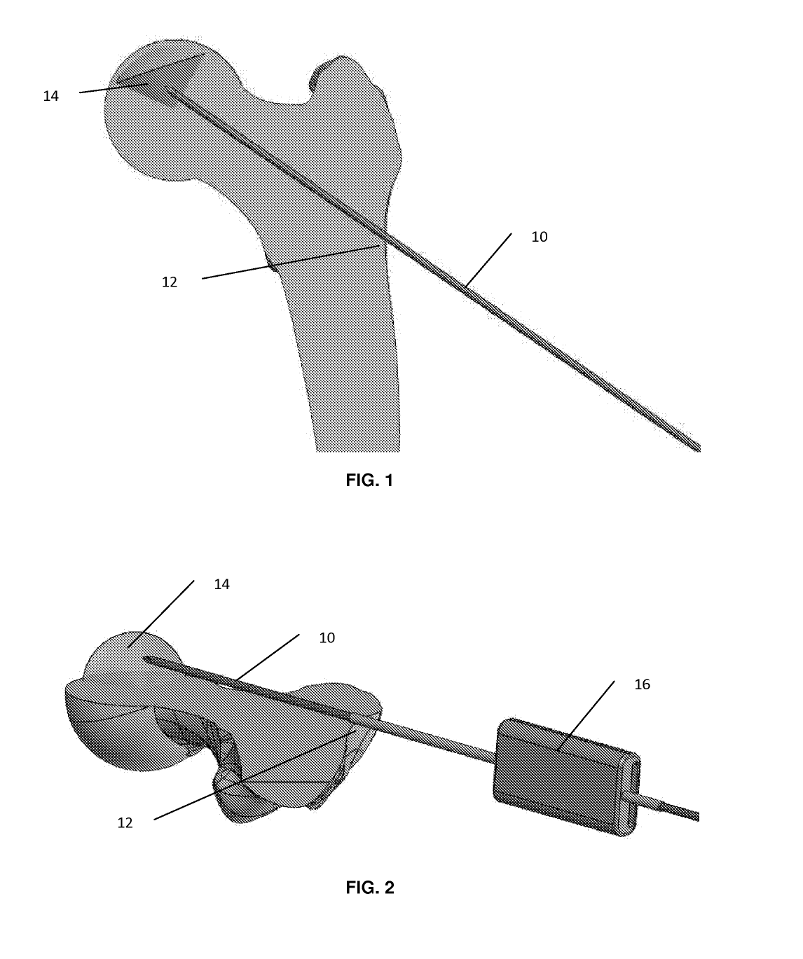

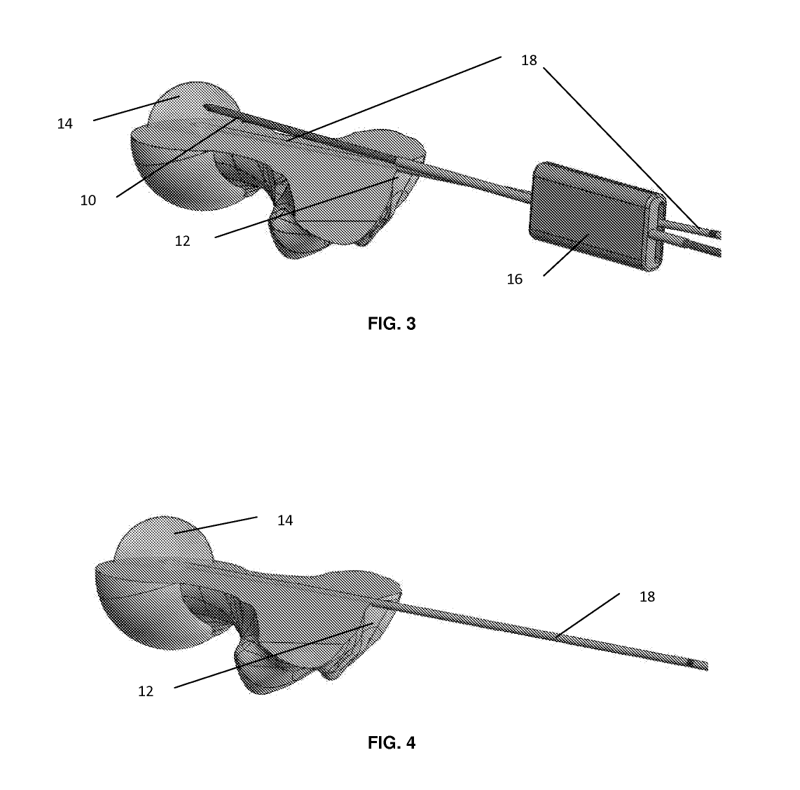

In more detail with reference to FIG. 1, the surgeon places a first guide-wire 10 under anterior-posterior (AP) fluoroscopic control through lateral cortex 12 and into necrotic bone 14. FIG. 2 shows the surgeon placing a three-dimensional guide 16 over the first guide-wire 10. FIG. 3 shows the surgeon placing a second guide-wire 18 through the slot in the three-dimensional guide 16. FIG. 4 shows the surgeon removing the first guide-wire 10 and three-dimensional guide 16, leaving the second guide-wire 18 in the selected (desired) position in the necrotic bone 14.

In another example of the approach, the surgeon places the guide-wire in the selected location completely under fluoroscopic control. The challenge with this approach is maintaining the trajectory of the guide-wire that was found acceptable in a first plane (e.g. AP or lateral), while redirecting it (or a second guide-wire) to an acceptable trajectory in a second plane (e.g. lateral or AP). The surgeon may need to continue to optimize placement of the guide-wire through continuous toggling between AP and lateral views (i.e., taking multiple orthogonal fluoroscopy images), losing alignment in one plane while adjusting the alignment in the other plane. This adds time to the procedure, and increases the radiation dose to the patient, surgeon, and supporting staff.

The three-dimensional guide 16 allows the surgeon to hold the proper orientation of the guide wire in one plane (e.g. AP or lateral) while adjusting the position in the other plane (e.g. lateral or AP). One can readily appreciate that contrasted with the “freehanded” approach described immediately above, using the three-dimensional guide 16 can reduce the number of fluoroscopy images taken and, thus, lessens the radiation dose to the patient, surgeon, and supporting staff.

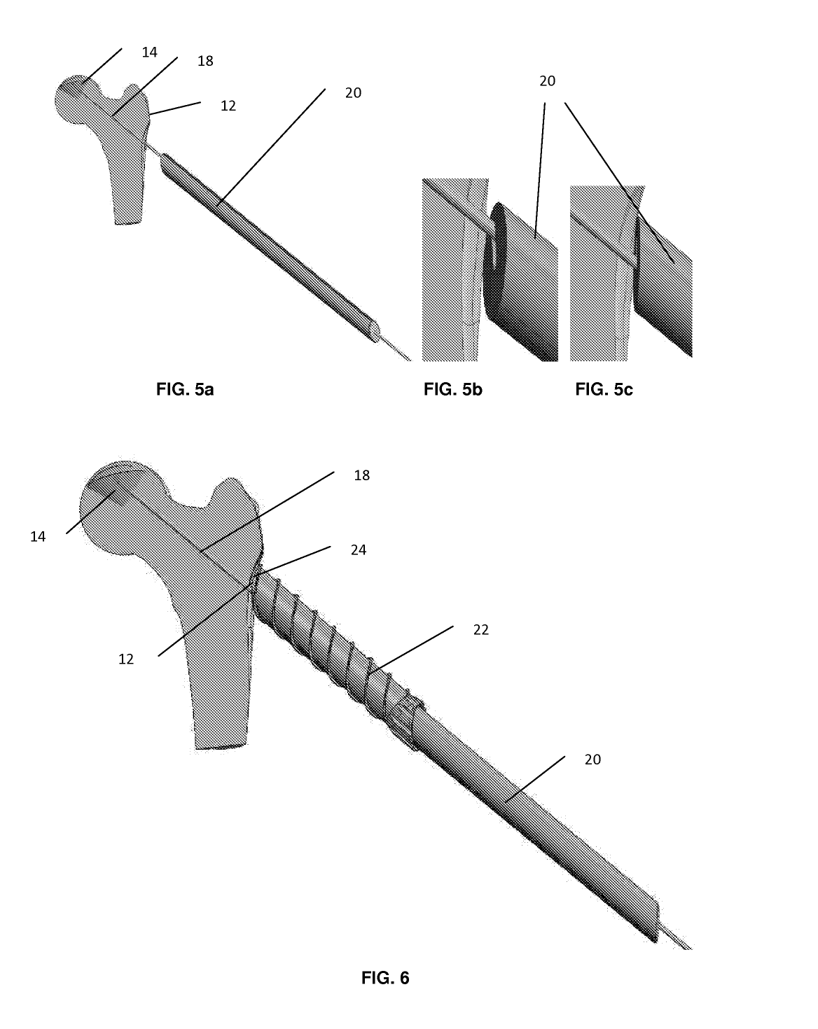

FIGS. 5a-5c show one example of the approach in which the surgeon creates a skin incision and then inserts an obturator 20 up against the lateral cortex 12, displacing soft tissue away from the bone entry site. The elongated cannulation in the obturator allows the obturator to be moved radially away from the guide-wire 18. The tip of the angled obturator is sharp, thus the radial movement allows the sharp tip to scrape aside the thin bone covering periosteum. In another example, the surgeon uses a standard (Cobb) elevator to push aside the thin bone covering periosteum in the area of the guide-wire 18.

FIG. 6 shows the surgeon inserting a skin cannula 22 with an angled tip 24 over the obturator 20 until the angled tip 24 is against and substantially parallel to the lateral cortex 12. The surgeon then removes the obturator 20. It should be readily apparent that the surgeon can insert a cannula having a variety of geometries and can use a variety of techniques to position the cannula. In a convenient example of the approach, the surgeon creates a skin incision and bluntly dissects soft tissue down to the femoral bone surface without the use of an obturator. The surgeon then spreads the soft tissue apart with standard tissue retractors (Hohmann) instead of inserting a cannula.

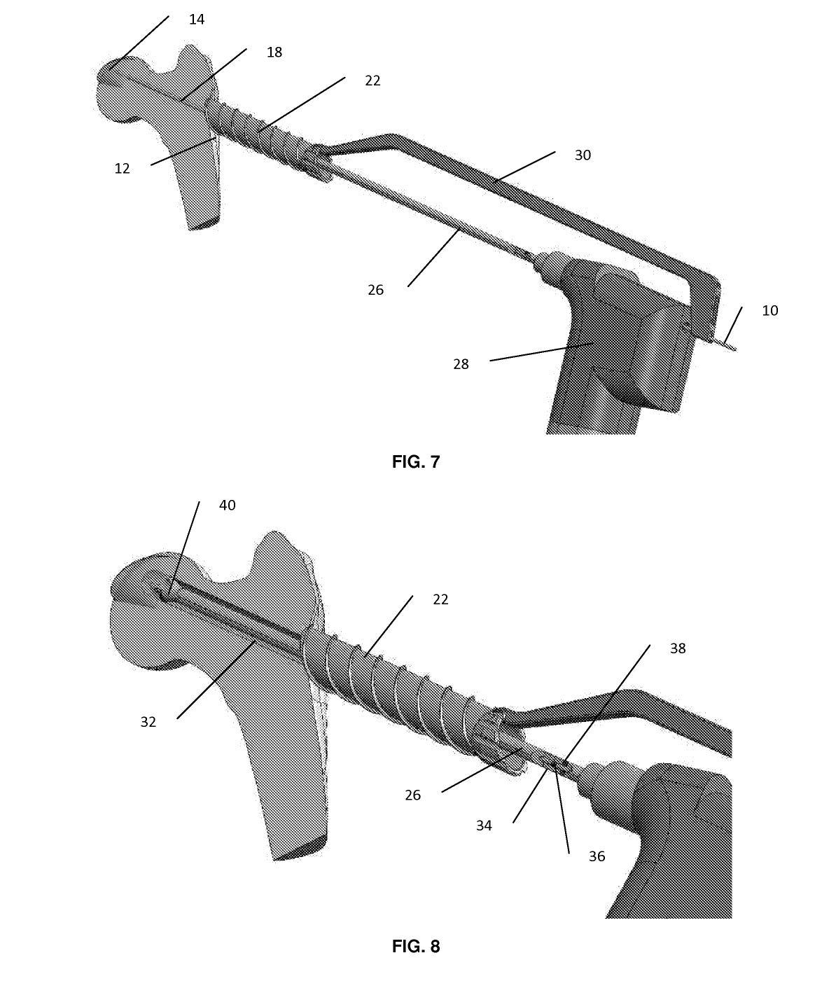

FIG. 7 shows a cannulated (core) drill bit 26 attached to and driven by a power drill 28. The surgeon slides the bit 26 over the free end (proximal end) of the guide-wire 18. The surgeon continues to pass the free end of the guide-wire 18 through the power drill 28 and out the back. The surgeon locks the position of the guide-wire 18 with a guide-wire locking arm 30. As shown, a first end of the guide-wire locking arm 30 attaches (or is integral) to the cannula 22. In another example, a first end of the guide-wire locking arm 30 abuts the lateral cortex 12, directly. A second end of the guide-wire locking arm 30 clamps or otherwise holds the free end of the guide-wire wire 18 (with or without use of a cannula). The guide-wire locking arm 30 is rigid and resists the tendency of the guide-wire 18 to move distally and deeper into the bone as the cannulated drill bit 26 advances over the guide-wire 18. This is beneficial because it eliminates or at least reduces the possibility of the surgeon penetrating through the femoral head with the guide-wire 18.

FIG. 8 shows the surgeon drilling the bone tunnel 32 to a predetermined depth. The drill bit 26 and guide-wire 18 include depth marks 36, 38 at their respective proximal portions, which the surgeon can see. The drill bit 26 further includes a long window 34 allowing the surgeon to see the depth mark 36 on the guide-wire 18 approaching and eventually lining up with the corresponding depth mark 38 on the drill bit 26. When the depth marks 36, 38 are aligned, the drill bit 26 is at the predetermined depth relative to the guide-wire 18 (e.g., drill tips are flush) thus preventing overdrilling and blow-out. As shown, the drill bit 26 incorporates a spherical end 40 to minimize stress concentrations between the tunnel end and subchondral bone. The surgeon then removes the guide-wire locking arm 30. In an example in which the guide-wire locking arm 30 is attached to the cannula 22, the surgeon breaks a one-time break-off joint on the guide-wire locking arm 30.

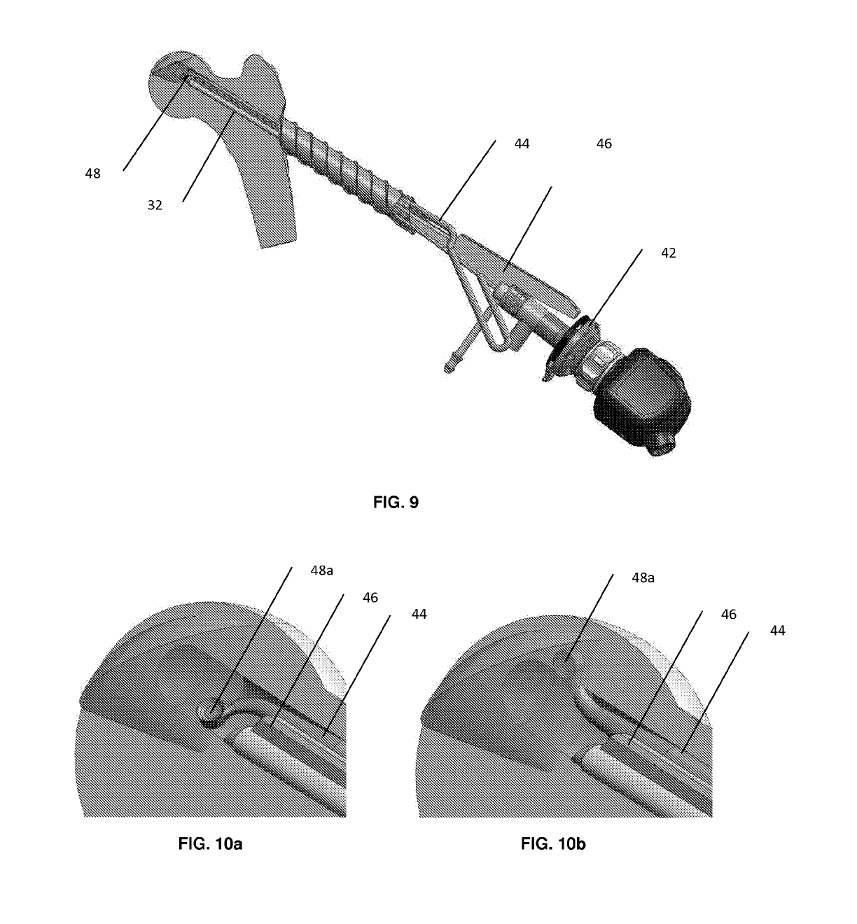

Turning now to a description of removing necrotic bone, FIG. 9 shows the surgeon inserting a visualization device 42, bone removal tool 44, and sheath 46 into the bone tunnel 32. In the following examples, the visualization device 42 is described and shown as being an endoscope (arthroscope). It is should be readily apparent that the visualization device 42 is not limited to an endoscope but include others, such as a camera (described later in greater detail). Continuing with FIG. 9, the sheath 46 holds the endoscope 42 and bone removal tool 44 together to form an assembly. The bone removal tool 44 has an axis of rotation about which a working end 48 of the bone removal tool 44 rotates. In some examples of the bone removal tool 44, the working end (tip) is bent or asymmetrical. The axis of rotation of the bone removal tool 44 is offset from the centerline of the bone tunnel 32 by the sheath 46. Because of the offset, it can be said that the axis of rotation of the bone removal tool 44 is on one side of the centerline of the bone tunnel 32.

FIG. 10a shows the surgeon inserting the assembly with the working end, shown as a bidirectional rotary curette 48a, on a side of the centerline opposite the side with its axis of rotation. FIG. 10b shows the surgeon removing bone by rotating the bone removal tool 44 (either manually or by using a power drill) so that the working end 48a is on the same side of the centerline as its axis of rotation. In some examples, the surgeon clears the resulting debris by irrigating the bone tunnel 32 with a fluid (liquid or gas).

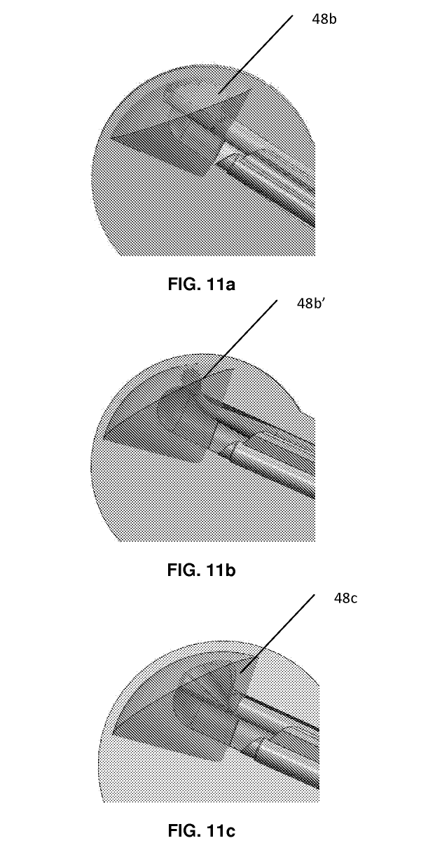

It should be readily apparent that in other examples of the approach, the surgeon may use bone removal tools with any number of different types and sizes of working ends. For example, FIGS. 11a and 11b show the surgeon using a manual rotary rasp(s) 48b and 48b′, respectively, to remove sclerotic bone. For more aggressive bone removal, FIG. 11c shows the surgeon using a single flute drill 48c.

While the surgeon is removing the necrotic bone, the surgeon can see the material being removed at the same time using the endoscope 42. Advantageously, removing bone under direct visualization, as described above, provides the surgeon with real-time visual feedback. The surgeon adjusts the bone removal process (e.g., remove more or less bone) in response to what the surgeon sees. Additionally, the approach also allows for direct visualization of the underside of the cartilage layer covering the femoral head. This is beneficial because such visualization prevents or at least minimizes the undesirable chance of breaking through the femoral head.

The prior approach requires the surgeon to stop removing bone (and possibly remove a bone removal tool from a bone tunnel) in order to insert an endoscope to inspect the progress and then to restart the process. The discontinuous nature of the prior approach makes the procedure tedious and time consuming Additionally, the prior approach requires the surgeon to remember what the surgeon saw and then remove bone based on that memory. In contrast, the bone removal under direct visualization approach is continuous. The endoscope 42, coupled to the bone removal tool 44 by the sheath 46, remains in the bone tunnel 32 during the bone removal process. In turn, the procedure is less tedious, less time consuming, and does not require the surgeon to remember what the surgeon saw and then remove bone based on that memory.

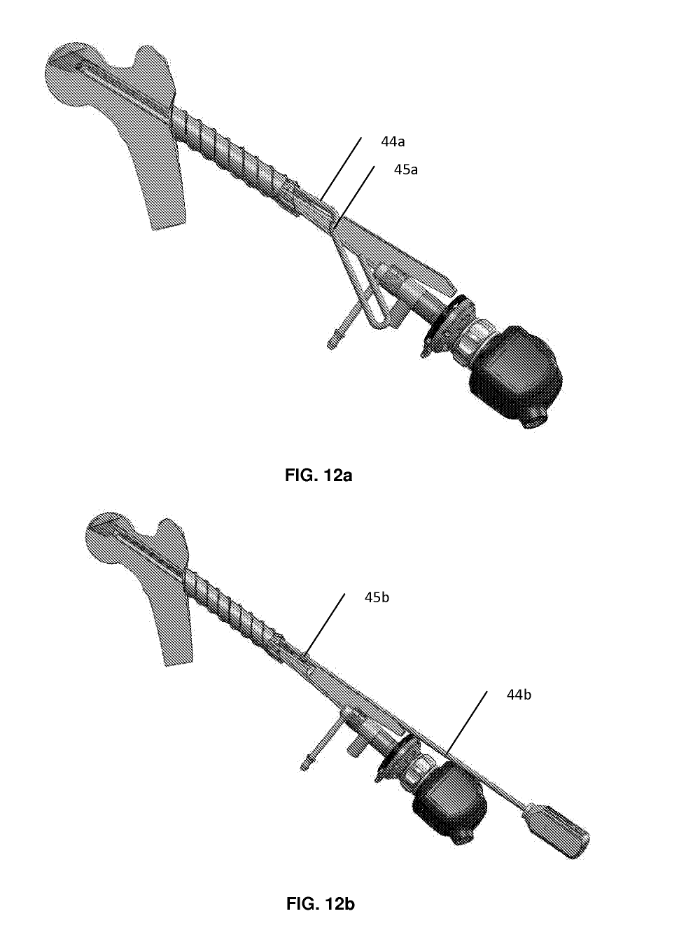

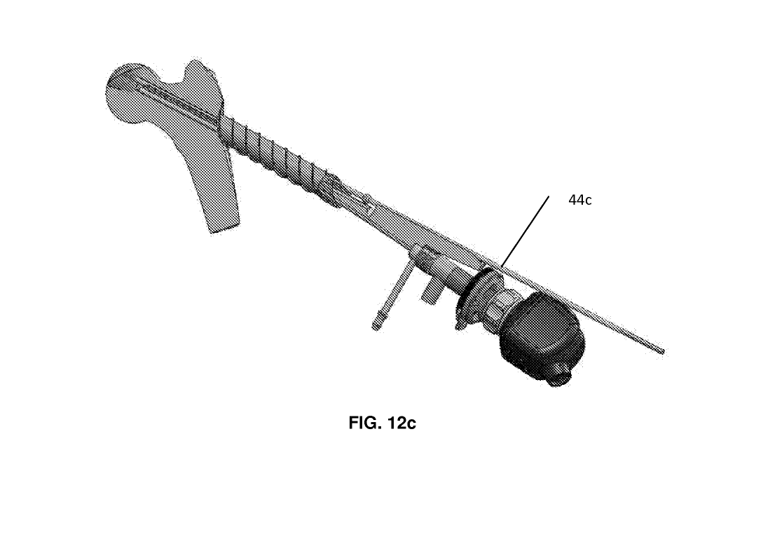

In some examples of the approach, the surgeon rotates the bone removal tool 44 manually. The manual examples provide the surgeon with tactile feedback. FIGS. 12a shows an example of the assembly with a short handled bone removal tool 44a. The short handled bone removal tool 44a includes a bend 45a in the shaft. The bend 45a cooperates with a stop in the sheath 46 (described later in greater detail). As shown, the short handled bone removal tool 44a runs part of the full length of sheath 46 and bends away from an axis of rotation to form a handle. FIG. 12b shows an example of the assembly with a long handled bone removal tool 44b. The long handled bone removal tool 44b includes a bead 45b formed around the shaft of the bone removal tool 44. The bead 45b cooperates with a stop in the sheath 46 (described later in greater detail).

In other examples of the approach, the surgeon rotates a powered bone removal tool 44c by using a powered device, such as power drill. In the example shown in FIG. 12c, a motor (e.g., electric, air or liquid) drives the powered bone removal tool 44c. In still other examples of the approach, the surgeon can use flexible instruments rather than rigid ones. These examples can reduce the diameter of the bone tunnel, which is desirable because less of the healthy bone is removed in forming the bone tunnel. The surgeon can use manual, flexible bone removal tools that allow for flexing of a distal shaft and allow for selective locking of that flex. The surgeon can use motorized burrs that allow for distal shaft flex.

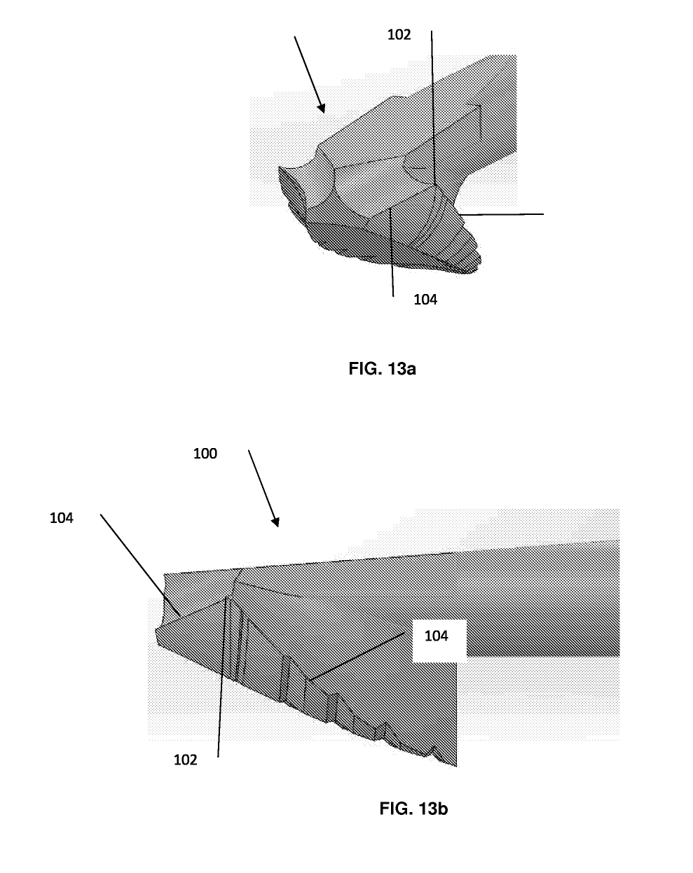

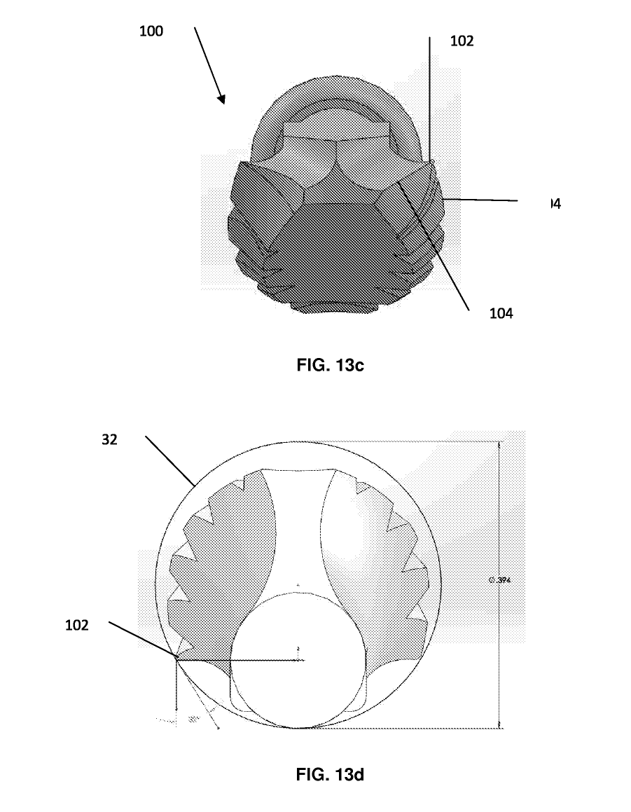

In addition to the examples of the working end 48 described above, another example of the bone removal tool 44 includes a rasp 100 with a three-dimensional geometry, as shown in the FIGS. 13a-13d. The three-dimensional rasp 100 includes a distinct leading point 102 that leads cutting edges 104. The cutting edges 104 sweep out to full diameter. The location of the leading point 102 is selected to minimize moment arm (and torque). As shown in FIG. 13d, the leading point 102 is located at the location of most challenging approach angle (e.g.,) 32°. In rotating the three-dimensional rasp 100, the leading point 102 contacts bone first. Subsequently, as the rasp 100 rotates, the cutting edges 104 engage and cut the bone.

Some examples of the bone removal tool 44 are offered in a series of steps, increasing the cutting size so as to minimize the torque required to turn them. Other examples of the bone removal tool 44 expand to increase the cutting size, again, to minimize the torque required to turn them. In still other examples, a combination of drill bit and bone removal tool forms a bone tunnel with a distal tunnel shape with minimum stress concentrations, i.e. , a continuous, curved surface. This is contrasted with a traditional drill bit that leaves a distinct edge between the distal conical face and cylindrical hole of the bone tunnel.

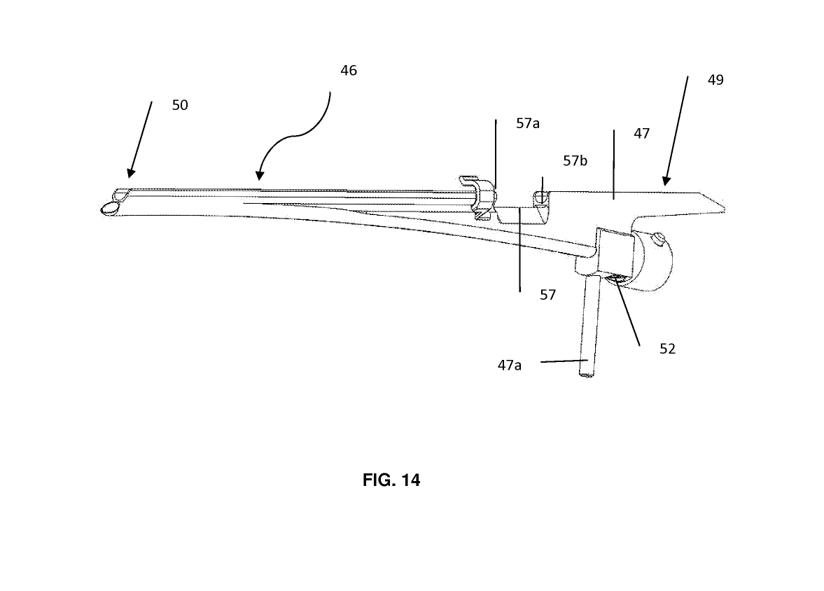

FIG. 14 shows an example of the sheath 46 that is used in conjunction with the visualization device 42 (of FIG. 9) and bone removal tool 44 (of FIG. 9) to remove bone through a bone tunnel. The sheath 46 includes a body 47 having a proximal end 49 and distal end 50. The distal end 50 of the body 47 is inserted into the bone tunnel. The sheath 46 also includes an inlet 52 disposed at the proximal end 49 of the body 47 (described later in greater detail). A working length of the body 47 is defined as a portion of the body 47 having a diameter smaller than the diameter of a bone tunnel. Put simply, the working length of the body 47 is what can fit inside of the bone tunnel.

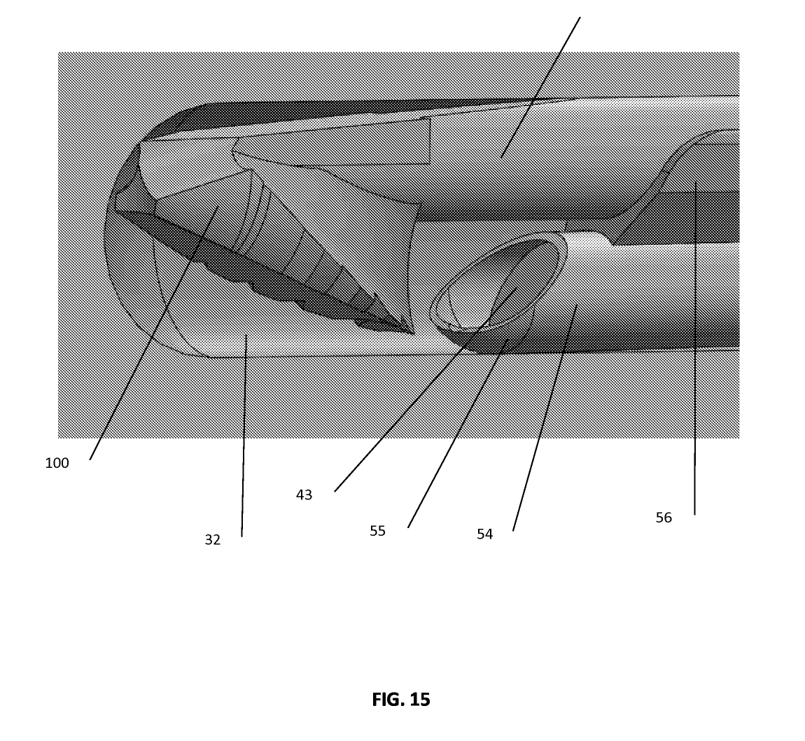

As shown in FIG. 15, the body 47 includes a first passageway 54 and a second passageway (working channel) 56, each extending, longitudinally, between the proximal and distal ends 49, 50 of the body 47. In the example shown, the first passageway 54 is configured to (slidably) receive a shaft of an endoscope with lenses 43 of the endoscope at the distal end 50 of the body 47. Some examples of the body 47 include a camera at the distal end 50 or at a distal terminus, which advantageously makes the body 47 smaller and able to fit into a bone tunnel with a smaller diameter. One“chip-on-a-stick”example has the camera formed, integrally, with the sheath 46. In another example, the camera is a separate element coupled to the sheath 46. The second passageway 56 is configured to (slidably and rotatably) receive the bone removal tool 44 with a working end (shown as the three-dimensional rasp 100 described above with reference to FIG. 13) at the distal end 50 of the body 47. The second passageway defines an axis of rotation of the bone removal tool 44.

As further shown in FIG. 15, the first and second passageways 54, 56 are spaced apart such that the axis of rotation of the bone removal tool 44 is offset from the centerline of the bone tunnel 32. As described above, because of the offset, it can be said that the axis of rotation of the bone removal tool 44 is on one side of the centerline of the bone tunnel 32. In insertion mode, a working end of the bone removal tool 44 is on a side of the centerline opposite the side with the axis of rotation. In bone removal mode, the working end of the bone removal tool 44 is on the same side of the centerline as the axis of rotation.

In some examples of the sheath 46 that are used in conjunction with an endoscope, the first passageway 54 (endoscope passageway) is curved to accommodate the relatively larger camera portion at the proximal end of the endoscope. The first passageway 54 and second passageway 56 are spaced apart a first distance at the distal end 50 of the body 47 and are spaced apart a second distance greater than the first distance at the proximal end 49 of the body 47.

In a convenient example of the sheath 46 for use with an endoscope shown in FIG. 15, a distal terminus 55 of the first passageway 54 enclosing the endoscope is rounded. Advantageously, this geometry minimizes bone from being scraped off of the tunnel wall 32 and blocking the fluid inflow or the field of view of the endoscope. This geometry is further advantageously because the rounded distal terminus 55 physically protects the optics 43 of the endoscope, which is both fragile and expensive to replace. In some examples, the endoscope is rotationally fixed within the first passageway. In other examples, the endoscope is rotatable within the first passageway 54. Rotating the endoscope changes the orientation of the field of view of the endoscope. For example, rotating a 30° field of view clockwise. This is beneficial because objects previously outside of the field of view and not seen by the surgeon can now be seen by rotating the endoscope.

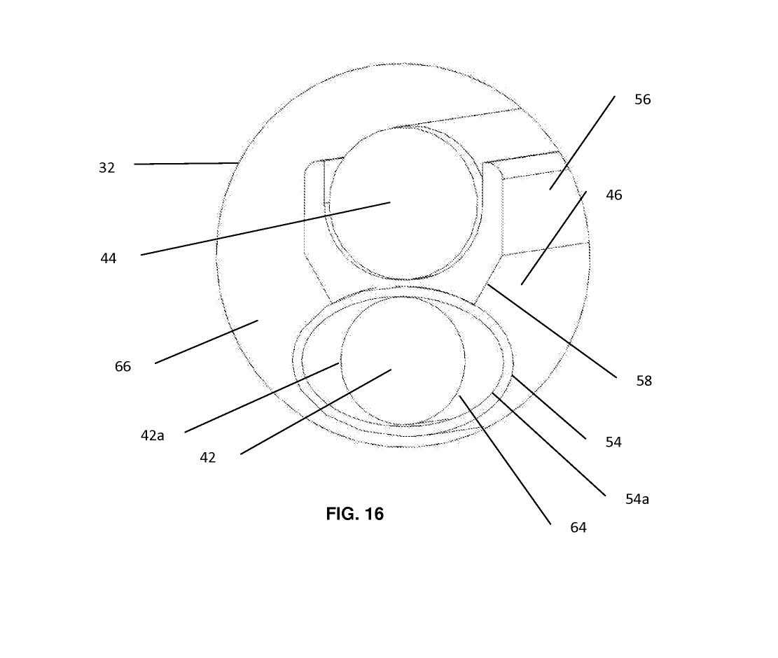

FIG. 16 shows the distal end of an assembly including the endoscope 42, bone removal tool 44, and a convenient example of the sheath 46 inserted into the bone tunnel 32. As shown, the second passageway 56 (bone removal tool passageway) is open on one side (i.e., a trough) making it easier to load and unload the bone removal tool 44. This is beneficial because a surgeon may use several bone removal tools with different working ends during the procedure. The second passageway 56 may be straight or curved depending on whether the bone removal tool 44 is manually or motorized. For example, a straight trough allows the use of motorized bone removal tools (e.g., the powered bone removal tool 44c of FIG. 12c). Some examples of the sheath 46 have the second passageway 56“above”the first passageway 54, as shown, in an arrangement that is aligned with the inlet 52 (of FIG. 14). Other examples of the sheath 46 have the second passageway 56“below”or lateral to the first passageway 54.

In a convenient example, an outer surface 42a of the endoscope 42 and an inner surface 54a of the first passageway 54, which houses the endoscope 42, form a conduit 64 for inflow fluid (liquid or gas). As shown, the endoscope 42 has a circle-shaped cross-section and the first passageway 54 has an oval-shaped cross-section. The inflow fluid conduit 64 is formed by the “difference” between the two cross-sections. It should be readily apparent that in other examples, the inflow fluid conduit 64 is formed by the endoscope 42 and first passageway 54 having cross-sections of a variety of shapes with a difference between the cross-sections.

In some examples, an outer surface of the body 47 and the wall of the bone tunnel 32 form an outflow conduit. For example, an inclined wall 58 formed in the body 47 and extending between the first and second passageways 54, 56 forms an outflow conduit 66. The outflow conduit 66 conveys debris away from the surgical site during the bone removal procedure. This is helpful because it removes debris from the field of view of the endoscope 42 that would otherwise obstruct or at least limit the surgeon's view of the procedure. The inclined wall 58 also reduces the cross-section of the assembly. Advantageously, this geometry minimizes the possibility of debris blocking the outflow between the wall of the bone tunnel and along the side of the assembly. As such, this clog resistant example of the sheath 46 is suitable for procedures in which clogging is likely.

Returning to FIG. 15, the sheath 46 includes the inlet 52 disposed at the proximal end 49 of the body 47. Inflow fluid (liquid or gas) is provided through the inlet 52 to push (flush) particulate (debris) away from the distal end 50 of the body 47 and push bone debris proximally out of the bone tunnel.

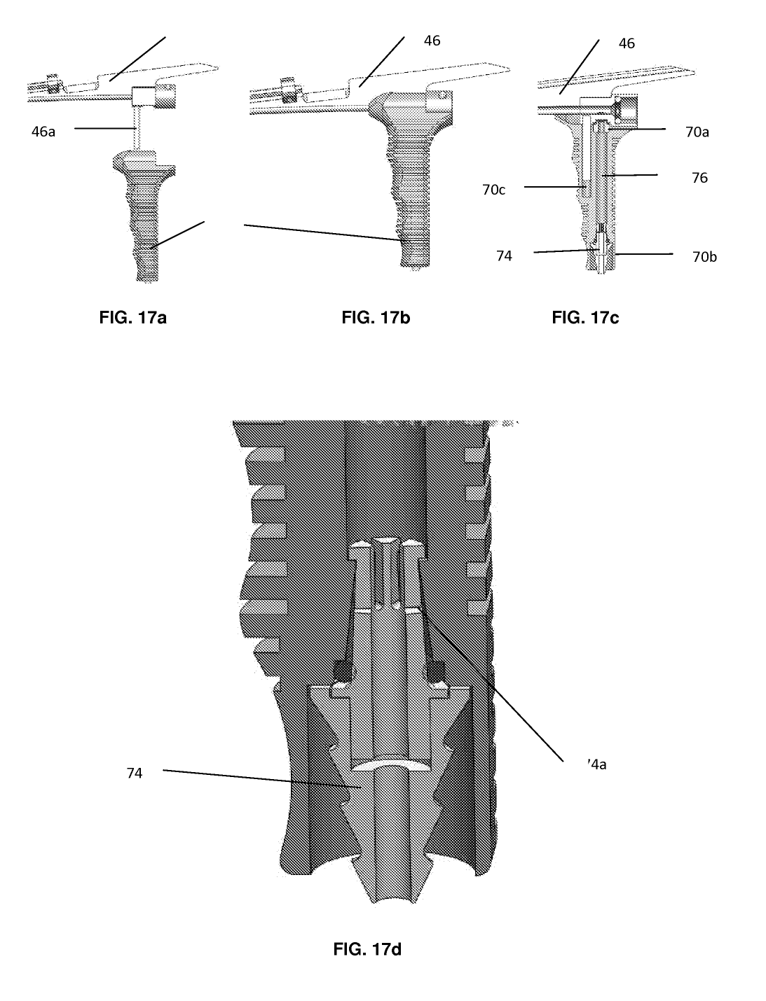

FIGS. 17a-17d show a convenient example in which the sheath 46 is connected to a source of the inflow fluid by an inlet port 70, which is in the form of a handle suitably shaped for holding. The inlet port 70 includes a first end 70a adapted to mate with the inlet 52 of the sheath 46, a second end 70b, and a passageway 76 extending between the first and second ends 70a, 70b of the inlet port 70. The a second end 70b includes coupling member 74 (described in greater detail below). When assembled together, the inlet 52 and the inlet port 70 are in fluid communication with one another and form a continuous passageway for the inflow fluid from the coupling member 74 to the distal end 50 of the sheath 46.

As best seen in FIGS. 17a and 17c, an example of the sheath 46 and inlet port 70 are mechanically joined together with an alignment pin 46a and alignment hole 70c. In this configuration, the sheath 46 is reusable and the inlet port 70 is disposable (e.g., provided in a disposable kit). Advantageously, this combination of reusable and disposable components promotes cleanliness and patient safety while reducing waste.

To further enhance the disposable nature of the inlet port 70, some examples of the coupling member 74 include a breakaway feature 74a that is best seen in FIG. 17d. The breakaway feature 74a causes the coupling member 74 to break apart when disconnecting the inlet port 70 from the inflow fluid source. This renders the inlet port 70 inoperable for repeated use. In the example shown in FIG. 17d, the coupling member 74 is a barb-type fitting. The barb retains an inflow tube conducting the inflow fluid from the source. The barb breaks if excess tension is applied, such as when the inflow tube is removed e.g., after surgery is complete. This disposable, single-use inlet port is beneficial to ensuring cleanliness and patient safety.

Returning to FIG. 14, an example of the sheath 46 includes a stop 57 that cooperates with a corresponding stop on the bone removal tool 44. This arrangement inhibits the bone removal tool 44 from moving too far in the direction of the proximal end 49 of the body 47 and damaging the tip of the endoscope 42. In a convenient example, the stop 57 is integrally formed with the body 47. The integrally formed stop 57 includes a first stop surface 57a and a second stop surface 57b (e.g., defining a notch). As best seen in FIGS. 12a and 12b, the first and second stop surfaces 57a, 57b cooperate with the bend 45a or bead 45b of the bone removal tool 44.

In another example, the stop 57 is formed as a protrusion extending from the second passageway and away from the body. The protrusion cooperates with the bend 45a in the shaft of the bone removal tool 44. In yet another example, the sheath 46 includes an indicator and the bone removal tool 44 includes a depth mark that the surgeon can see. When the depth mark on the bone removal tool 44 lines up with the corresponding indicator on the sheath 46, the surgeon knows the bone removal tool 44 is close to the endoscope and moving past the mark will likely damage the endoscope. In still yet another example, the sheath 46 incorporates a selectively lockable mechanism to retain the bone removal tool 44 within the second passageway 56.

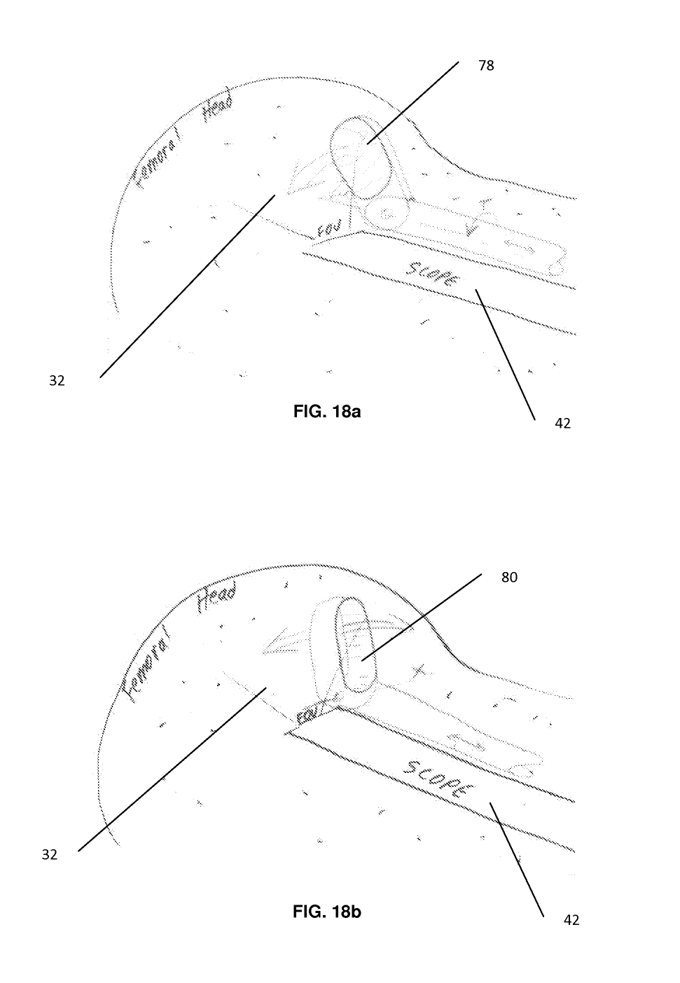

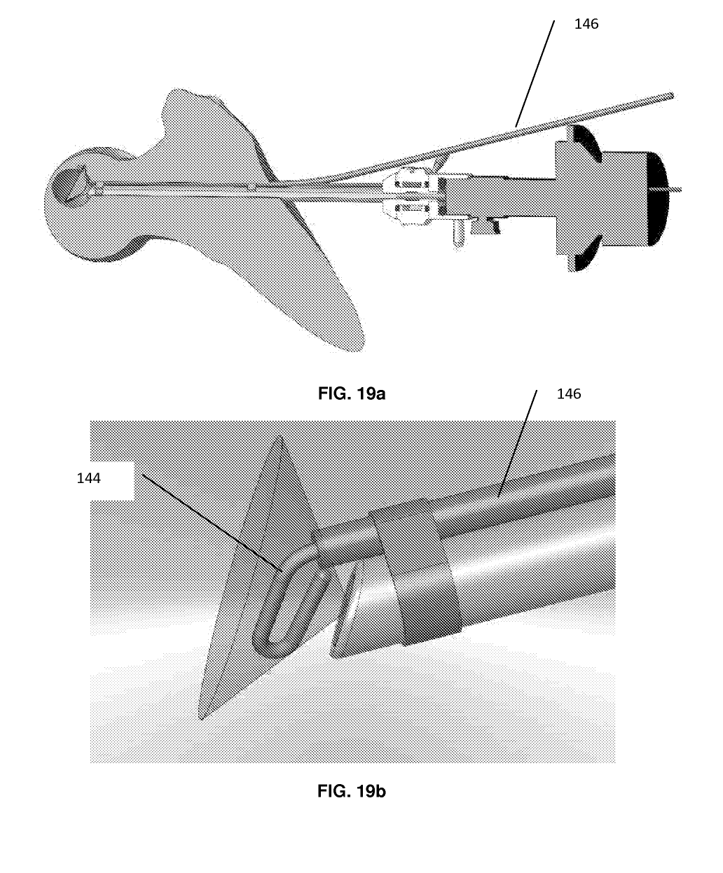

The foregoing discussion describes examples of the bone removal under direct visualization approach in the context of using an assembly including a sheath. Other examples of the approach are described below. FIGS. 18a and 18b show a surgeon using an articulating rotary curette 78 and an articulating planer curette 80, respectively, to dislodge and evacuate necrotic bone out of a bone tunnel 132. FIG. 18a shows the surgeon inserting the articulating rotary curette 78 into the bone tunnel 132 with the angle of a working end set to 0° (i.e., aligned with the centerline of the bone tunnel 132). The surgeon inserts the articulating rotary curette 78 and endoscope 142 together. (The endoscope 142 having a field of view (FOV).) The surgeon flexes the articulating rotary curette 78 so that the angle of the working end is greater than 0° (i.e., above the centerline of the bone tunnel 132). The surgeon rotates the articulating rotary curette 78 (and pistons the articulating rotary curette 78) to dislodge and evacuate the necrotic bone out of the bone tunnel 132. The articulating rotary curette 78 may be closed (as shown) or opened.

FIG. 18b shows the surgeon inserting the articulating planer curette 80 into the bone tunnel 132 with the angle of the working end set to 0° (i.e., aligned with the centerline of the bone tunnel 132). The surgeon inserts the articulating planer 80 and endoscope 142 together into the bone tunnel 132. (The endoscope 142 having a field of view (FOV).) The surgeon flexes the articulating planer curette 80 so that the angle of the working end is greater than or less than 0 degree (i.e., above or below the centerline of the bone tunnel). The surgeon rotates the articulating planer curette 80 as well as pistons the articulating planer curette 80 to dislodge and evacuate necrotic bone out of the bone tunnel 132. The articulating planer curette 80 may be closed or opened (as shown). The articulating planer curette 80 may be rigid, flexing; motorized or manual. In some examples, the movable (articulating) curette head is biased to an angled position (e.g., using a Nitinol wire) or actively positioned (e.g., pull-pull cables/pull rod).

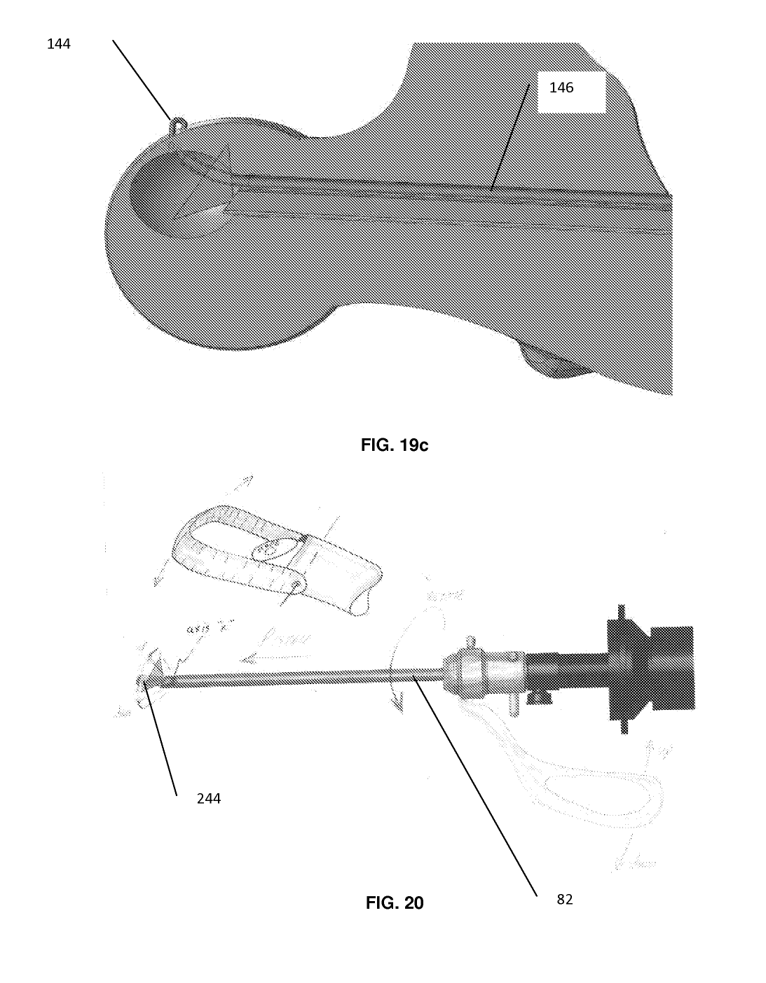

FIG. 19a-19c show another example of the approach in which a surgeon using a rotary wireform 144 to dislodge and evacuate necrotic bone out of a bone tunnel. The surgeon inserts the rotary wireform 144 alongside an endoscope or concurrently until the wireform is within the field of view of the endoscope. The rotary wireform 144 is flexible and/or expanding and is designed to break up the necrotic bone without damaging the flexible cartilage layer. The surgeon spins the rotary wireform 144 (either manually or by using a power drill) and pistons the rotary wireform 144 within the field of view of the endoscope. The surgeon may bias and steer the rotary wireform 144 via a curved tube 146, for example, as shown. Some examples of the rotary wireform 144 concept are incorporated into an integral endoscope sheath. Examples of an integral endoscope sheath are described immediately below.

FIG. 20 shows another example of the approach in which a surgeon uses an endoscope sheath 82 with an integral flexing bone removal tool 244 (shown in the figure as an articulating planer curette actuated by a handle 146) to dislodge and evacuate necrotic bone out of a bone tunnel under direct visualization. The integral sheath 82 includes a body having a proximal end and a distal end. The body defines a passageway extending, longitudinally, between the proximal and distal ends, and has an axis. The passageway is configured to receive the endoscope.

The integral flexing bone removal tool 244 is disposed at the distal end of the body. In the example shown, the integral flexing bone removal tool 244 flexes about an axis substantially perpendicular to the axis of the endoscope passageway.

In the foregoing examples of the integral sheath 82, an axis of rotation of the bone removal tool 244 and the axis of the endoscope passageway, and, thus, the endoscope are axially aligned or coaxial. Bone removal is within the field of view (FOV) of the endoscope. This approach allows the surgeon to actuate the endoscope and bone removal tool with one hand (e.g., by way of a handle, as shown). Some examples of the integral sheath 82 are disposable.

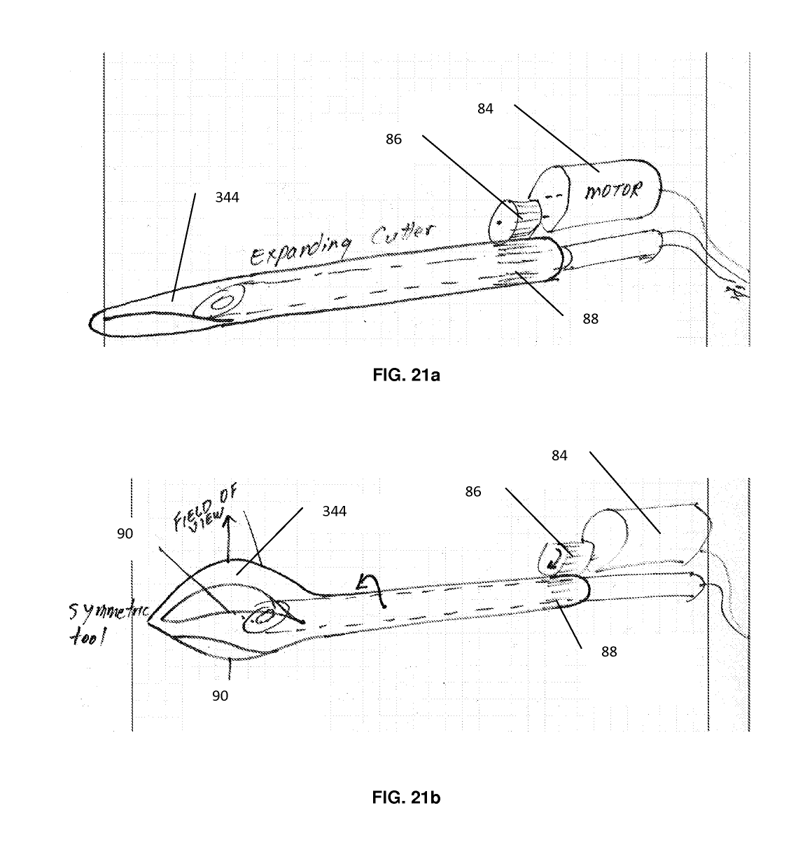

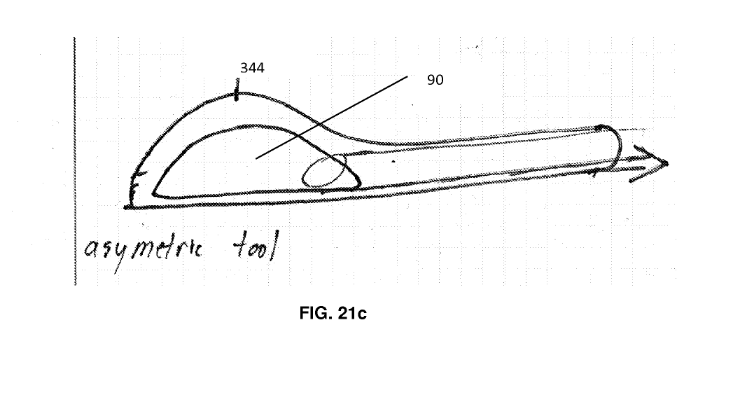

FIGS. 21a and 21b show another example of the integral sheath 82 with an expanding bone removal tool 344 that expands in a direction substantially perpendicular to the axis of the endoscope passageway. For example, the expanding bone removal tool 344 expands radially outward from a first diameter to a second diameter (and diameters in between). A motor 84 drivingly coupled to the proximal end of the body rotates the body and expanding the bone removal tool 344 about the axis to remove bone. As shown, but in no way limiting, a pinion gear 86 is mounted to the motor 84. The pinion gear 86 cooperates with an annular gear 88 formed circumferential at the proximal end of the body. Rotating the pinion gear 86, in turn, rotates the body and the expanding bone removal tool 344. Those skilled in the art will readily recognize other drive mechanisms are possible to rotate the body and the expanding bone removal tool 344. In another example, the body rotates the manually. The surgeon applies a manual torque to rotate the body and the expanding bone removal tool 344.

The endoscope remains fixed as the body and expanding bone removal tool rotate 344 around the endoscope. In a convenient example, an inflow liquid or gas passes between the outer surface of the endoscope and the inner surface of the passageway to reduce rotational friction between the endoscope and body. The inflow liquid or gas also keeps the site clear of debris, as described above.

The expanding bone removal tool 344 has a proximal end and a distal end. One or more expansion slits 90 run between the proximal and distal ends of the expanding bone removal tool 344. The expansion slit 90 is at a selected angle relative to the axis of the endoscope passageway. In some examples, the selected angle is 0° i.e., the expansion slit 90 is parallel to the axis of the endoscope passageway. The form, number, angle, and length of the expansion slit 90 are selected to provide an expanding bone removal tool 344 suitable for removing bone under direct visualization. In an expanded state, the expansion slit 90 opens and the bone removal expands to a diameter greater than the diameter of the bone tunnel. The resulting opening in the expansion slit 90 enables the surgeon to see the bone being removed. The expansion slit 90 is shown being a straight line in form but other forms are possible, such as a wave.

In the example of the integral sheath 82 shown in FIGS. 21a and 21b, the expanding bone removal tool 344 expands symmetrically about the axis. In another example of the integral sheath 82 shown FIG. 21c, the expanding bone removal tool 344 expands asymmetrically about the axis.

In some examples, the integral sheath 82 further includes an actuating means for expanding the expanding bone removal tool 344. Such means include push/pull rods, pull-pull cables, and an untwisting tube. In other examples, the expanding bone removal tool 344 expands as it is rotated (i.e., by centrifugal force).

Some curette and wireform examples do not need to be passable along the length of the endoscope. These examples may be permanently assembled in their working configuration. To the extent any of the foregoing examples include an actuating mechanism, such mechanism can take many forms from live hinges to push/pull rods to pull-pull cables to sprung curettes (the natural state of which is bent), just to name a few. Flexing instruments, such as a flexing, motorized arthroscopy burr or a selectively lockable, flexing curettes/rasps are suitable for removing bone laterally from a bone tunnel. It should be readily apparent that these flexing instruments may be used with the bone removal under direct visualization approach just described.

1. A sheath comprising:
a body including a proximal end and a distal end, the body further including:
a first passageway extending, longitudinally, between the proximal and distal ends of the body;
a second passageway extending, longitudinally, from the distal end of the body towards the proximal end of the body and defining an axis of rotation of a bone removal tool;
wherein the first and second passageways being spaced apart such that the axis of rotation of the bone removal tool is offset from the centerline of a bone tunnel;
wherein a working length of the body has a diameter smaller than the diameter of the bone tunnel; and
an inlet disposed at the proximal end of the body through which inflow fluid is provided.

2. The sheath of claim 1 wherein the first passageway is curved with the first passageway and the second passageway spaced apart a first distance at the distal end of the body and spaced apart a second distance greater than the first distance at the proximal end of the body.

3. The sheath of claim 1 wherein at the distal end of the body, the first passageway terminates with a rounded end.

4. The sheath of claim 1 wherein the second passageway is a U-shape trough.

5. The sheath of claim 1 wherein the first passageway and the second passageway are stacked on one another along a lateral axis defined by the inlet.

6. The sheath of claim 1 further comprising a stop integrally formed with the body; and
wherein the integrally formed stop includes a first stop surface and an opposed second stop surface, the first and second stop surfaces cooperate with a corresponding bead formed around a shaft of the bone removal tool or a corresponding bend formed in a shaft of the bone removal tool to limit movement of the bone removal tool along a length of the second passageway.

7. The sheath of claim 1 further comprising an inclined wall formed between the first passageway and the second passageway, the inclined wall defines a conduit with the wall of the bone tunnel for conducting outflow fluid carrying portions of removed bone.

8. A system comprising :
a sheath including:
a body comprising a proximal end and a distal end, the body further comprising:
a first passageway extending, longitudinally, between the proximal and distal ends of the body;
a second passageway extending, longitudinally, from the distal end of the body towards the proximal end of the body and defining an axis of rotation of a bone removal tool;
wherein the first and second passageways being spaced apart such that the axis of rotation of the bone removal tool is offset from the centerline of the bone tunnel;
wherein a working length of the body has a diameter smaller than the diameter of the bone tunnel; and
an inlet disposed at the proximal end of the body through which inflow fluid is provided; and
a visualization device received in the first passageway of the sheath.

9. The system of claim 8 wherein the first passageway of the sheath is curved with the first passageway and the second passageway spaced apart a first distance at the distal end of the body and spaced apart a second distance greater than the first distance at the proximal end of the body.

10. The system of claim 8 wherein the first passageway of the sheath and the visualization device have different cross-sections; and
wherein the difference in cross-sections defines a conduit for the inflow fluid.

11. The system of claim 8 wherein the visualization device is an endoscope.

12. The system of claim 8 further comprising a bone removal tool including a shaft and a working end at an end of the shaft; and
wherein at least a portion of the shaft of the bone removal tool is received in the second passageway of the sheath.

13. The system of claim 12 wherein the shaft of the bone removal tool is flexible.

14. The system of claim 12 wherein the working end of the bone removal tool includes a three-dimensional rasp comprising two cutting edges meeting at a leading point; and
wherein the leading point meets the wall of the bone tunnel at a 32° angle and contacts bone before the two cutting edges as the working end is rotated.

15. The system of claim 12 wherein the working end of the bone removal tool includes any one of rotary rasp, articulating rotary curette, articulating planer curette, and rotary wireform.

16. The system of claim 8 further comprising an inlet port including a first end adapted to mate with the inlet of the sheath and a second end adapted to mate with an inflow fluid source.

17. The system of claim 17 wherein the inlet port is in the shape of a handle.

18. The system of claim 17 wherein the second end includes a coupling member with a breakaway feature, such that when the second end of the inlet port is being disconnected from an inflow fluid source the coupling member breaks away from the second end.

19. A bone removal tool comprising:
a shaft having a length, a portion of which is supported by a passageway of a sheath; and
a working end at an end of the shaft.

20. The bone removal tool of claim 19 wherein the working end has an axis of rotation defined by a second passageway of the sheath and is offset from the centerline of a bone tunnel.

21. The bone removal tool of claim 19 wherein the shaft is flexible.

22. The bone removal tool of claim 19 wherein the shaft includes a bead formed around the shaft; and
wherein the bead cooperates with a first stop surface and an opposed second stop surface of a stop integrally formed with the sheath.

23. The bone removal tool of claim 19 wherein the shaft includes a bend formed in the shaft;
and wherein the bend cooperates with a first stop surface and an opposed second stop surface of a stop integrally formed with the sheath.

24. The bone removal tool of claim 19 wherein the working end of the bone removal tool includes a three-dimensional rasp comprising two cutting edges meeting at a leading point; and
wherein the leading point meets the wall of the bone tunnel at a 32° angle and contacts bone before the two cutting edges as the working end is rotated.

25. The bone removal tool of claim 19 wherein the working end includes any one of rotary rasp, articulating rotary curette, articulating planer curette, and rotary wireform.
